(19) United States
(12) Patent Application Publication
(10) Pub. No.: US 2008/0287768 A1
(43) Pub. Date: Nov. 20, 2008

(54) NECKLACE TYPE OF DETECTOR FOR ELECTROCARDIOGRAPHIC AND TEMPERATURE INFORMATION

(75) Inventors: Terry B. J. Kuo, Hualien County (TW); Cheryl C.H. Yang, Hualien County (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL YANG-MING UNIVERSITY, Taipei City (TW)

(21) Appl. No.: 11/832,157

(22) Filed: Aug. 1, 2007

(30) Foreign Application Priority Data
May 15, 2007 (TW) 096117164

(51) Int. Cl. A61B 5/0408; A61B 5/01

(52) U.S. Cl. 600/382; 600/509; 600/549

(57) ABSTRACT

The present invention provides an non-adhering electrocardiographic and temperature signal detector comprising: (a) an electrocardiographic detecting working electrode, an electrocardiographic reference electrode and a temperature sensor connected together by a conduction chain; (b) a signal processing unit, for processing those signals obtained from said electrode or sensor, (c) a wireless transceiver for sending and receiving processed signal of electrocardiographic to the receiver at a far end unit or receiving data from the tar end unit; and (d) a power supply supplying power to the operation of the detector. The detector improves the uncomfortable of conventional adhering detector and increase the willing of a use due to the aesthetic, outlook of the detector and convenient to detect. Accordingly, the electrocardiographic and body temperature gas and opportunity to apply home care device.

### FIELD OF THE INVENTION

The present invention is related to the field of non-invasive biosignal measurement and more specifically to wireless mini-detector for electrocardiographic signal and body temperature measurement.

### DESCRIPTION OF RELATED ART

Generally, the research of medical science can be classified into two major areas, structuralism and functionalism; the former wants to know the composition of living organism and the relationship between each organ by anatomy, but the latter wants to study the function of each organ by detecting and measuring its' property it behaves.

Recently, the growth of functional medical study has a great progress; there are lots of techniques established for biosignal detection. But in order to get more accurate information, they usually adapt an invasive method for biosignal collection. For example, heart catch (also called cardiac catheterization), a method shows blood vessels of the heart and the inside of the heart as it pumps, needs to put a catheter into artery then guided into the heart, since it can increase the accuracy for diagnosis but it also increases the risk during surgery and the patient should endure the pain. Comparatively, non-invasive method is much safe than invasive one, but due to the indirect detection method it can not get accurate enough signal for medical use.

Fortunately, with the aids of the development of computer science, someone can use powerful software and hardware to improve the signal processing ability of the non-invasive detector. Heart rate variability (HRV) analysis (Anonymous 1996, Circulation 93: 1043-1065.) represents a good example for non-invasive diagnosis, which detects the skin surface voltage variability and processes those signals to estimate of the function of the sympathetic and parasympathetic autonomic nervous systems in patients. Our group had successfully applied this technique on analysis of cardiovascular fluctuations during pentobarbital anesthesia (Yang et al. 1996, American Journal of Physiology 270:H575-H582.), brain death determining (Kuo et al. 1997, American Journal of Physiology 273: H1291-H1298.), prediction of patient outcome in an intensive care unit (Yien et al. 1997, Critical Care Medicine 25: 258-266.), and observation the effect of aging on gender differences in neural control of heart rate (Kuo et al. 1999, American Journal of Physiology 277: H2233-H2239.), but consider to the convenience of those non-invasive technique and the feeling of patients, there is still lots of work we can do.

The non-invasive diagnosis technique mainly is consisted of two parts, one is detector and the other is signal processing unit. The detector is the most important part in this field because without suitable detector, there is no accurate signal be get and the further signal procession is useless, or if the detector is hard to apply on patient then the patient will also feel uncomfortable as using the invasive one. Thus the key point to develop a non-invasive diagnosis technique is how the design a detector with accuracy, comfortable and user friendly properties.

Presently, the long-term physiological signal detection system is based on the wire-transmission technique. When one patient needs to use this system, he should be stick a lot of electrodes on his skin, and the signals collected by those electrodes will be transmitted through the connected wires to a signal processing unit to be further processed such as analog to digital conversion and signal amplification. Because it should take long time in collecting those wanted signals, the stick electrodes and connected wires would make patient uncomfortable and hindrance. Besides, due to the unfriendly interface of this system, it needs professional technician to operate the machine. Thus this type diagnosis system is hard to apply on daily life.

Recently, some companies developed new wireless systems to transport the detected signals. Those systems generally comprise three parts, one is detecting electrodes, another is a signal processor and the other is a far-end signal analysis/storage machine, wherein the electrodes detect physiological signals, deliver them through wires to the signal processor to process analog-to-digital conversion and amplification, and then transport those signals to the far-end signal analysis/storage machine. This system has been successfully applied on some clinical use and it gives a hint that wireless technique can apply on this area. However, this system still can not integrate the electrode and signal processor together but builds those two parts separately and needs wire to connect them, thus this limitation also increase some side-effect accompanied by the wires used such as noise and too heavy to carry.

Due to the great progress of diagnosis technology we can quantify the autonomic nervous activity through processing the electrocardiography (ECG) signals. In order to get more specific physiological signals most of the electrodes were used in a disposable patch-form which sticks to a patient's skin, but these sticky electrodes somehow cause skin allergy and the non-reusable electrodes will also increase the burden for user's daily use, thus the user may not want to keep using it for long-term care.

### SUMMARY OF THE INVENTION

In order to improve those defects said above, here we provides a novel non-stuck detector for electrocardiographic and body temperature information measurement. This detector designed in necklace-type wherein the pendant part has a tiny electrode and some necessary components such as signal processor and wireless transceiver in it, and the chain part is another tiny electrode. This detector is not only a physiological signal detector but also a well-designed accessory, and it is very easy for use as long as the user keeps steady posture and lets the detector fixed onto his skin for a short term thus the electrocardiographic signal and body temperature information can be got and then transmitted to far-end receiver for further analysis through a wireless transceiver in the pedant. So, this physiological signal detector is very useful for long-term care use.

### BRIEF DESCRIPTION OF THE DRAWINGS

FIG. 1 shows an embodiment of non-stuck detector for electrocardiographic and temperature information measurement, wherein the reference electrode is a conductive plate placed on the chain.

FIG. 2 shows an alternative embodiment of non-stuck detector for electrocardiographic and temperature information measurement, wherein the reference electrode is the chain itself.

FIG. 3 illustrates how the detector applied on a user.

FIG. 4 illustrates a diagram of the circuit design of the detector.

FIG. 5 illustrates a datasheet got from post-analyzing of the electrocardiographic and body temperature information by a far-end computer.

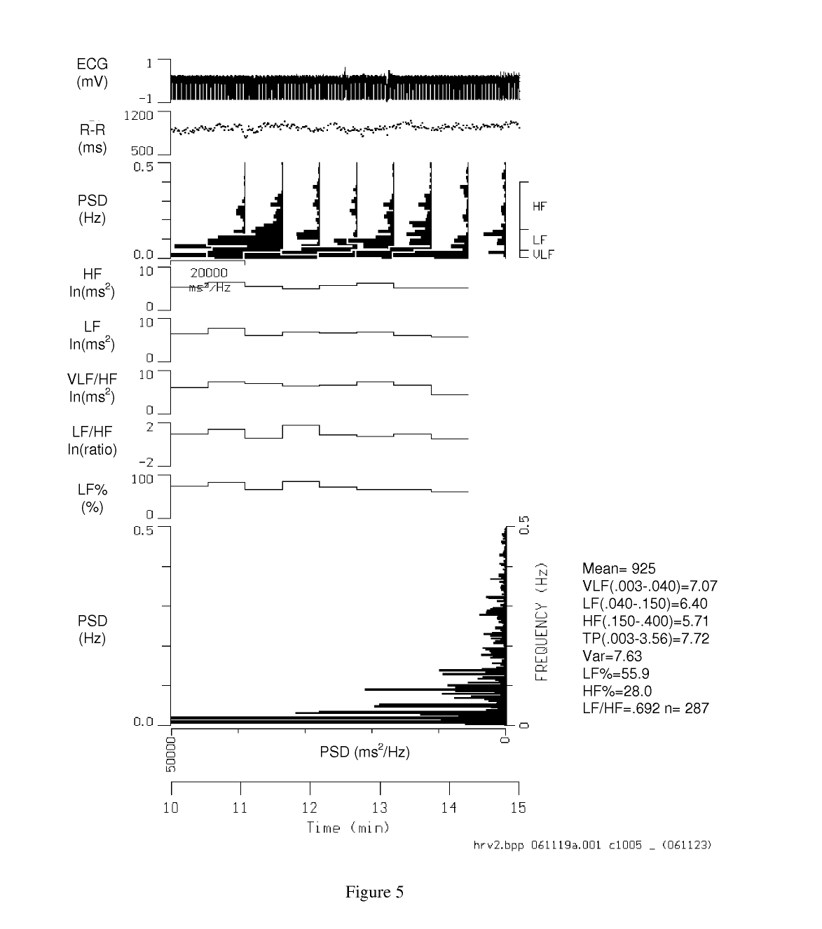

### DETAIL DESCRIPTION OF THE INVENTION

The electrocardiographic/body temperature signals detector of this invention comprises at least five components, which are (a) a working electrode and a reference electrode for electrocardiographic signals acquisition; (b) a temperature sensor for body temperature detection; (c) a signal processing unit for processing said physiological signals; (d) a wireless transceiver for connection with a far end machine such as a computer; and (e) a power supply, wherein the working electrode and the reference electrode were constructed into a two-electrode system, the former detects a surface voltage of an use's skin through its' induction coil and the latter provides a reference voltage, thus an electrocardiographic signal is got by comparing the surface voltage and the reference voltage. In this invention, the function of the reference electrode is to be a reference for electrocardiographic signals detection and amplification. The chain used in this invention contains a conducting wire in it and provides the connection between all the said components.

The working electrode and the reference electrode of this invention are placed separately on the conductive chain and on a pendant linked to said chain, wherein the electrode placed on the chain part is the chain itself or is a conductive plate on the chain, and the electrode placed on the pendant is a conductive outer shell of said pendant or is an induction coil underneath the shell. The said conductive outer shell is made by metal or by plastic having electrical conductivity, and the plastic is conductive polymer, or is polymer processed by electroplating or coating with conductive material. In the best embodiment, the working electrode is placed on the pedant and the reference electrode is a conductive plate placed on the chain.

The temperature sensor of this invention detects body temperature through resistance thermometer, thermistor, thermocouple, or any non-invasive electric-thermometer.

In this invention, the signal processing unit, the wireless transceiver and the power supply are preferred to place in the pendant with the working electrode, but they still can be placed separately in different pedants as long as they were linked by a same conducting wire. Further more, this invention may also have a data storage unit in the pedant for electrical signals storage.

The signal processing unit of this invention comprising: (a) a signal amplifier; (b) an electronic filter; and (c) an analog-to-digital converter (ADC), wherein the electronic filter is selected from the group consisting of low-pass filter, high-pass filter, band-pass filter, band-stop filter and all-pass filter.

The wireless transceiver of this invention transports signals through radio wave interface or infrared interface, wherein the radio wave interface is IEEE 802 standard, RFID, GSM, PHS, CDMA or any other radio wave interface that can be used for electronic signal transmission.

The power supply of this invention is to provide electric power for all the components working, which may be a solar cell or a chemical battery, and the preferred one is using chemical battery.

The present invention is applied on a user's neck, wrists, or limbs, but the preferred embodiment is applied on the neck.

### EXAMPLE

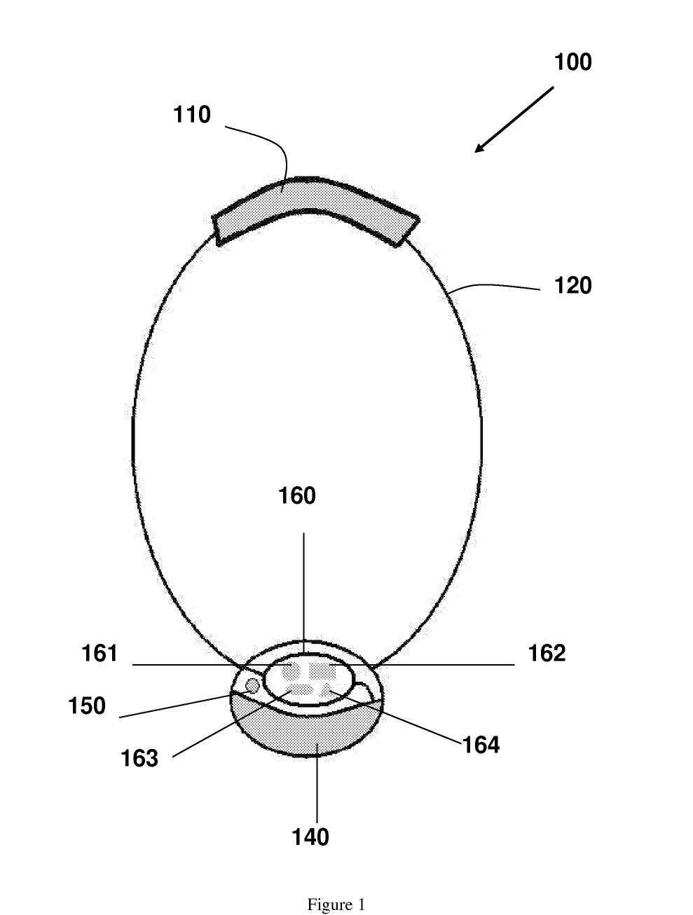

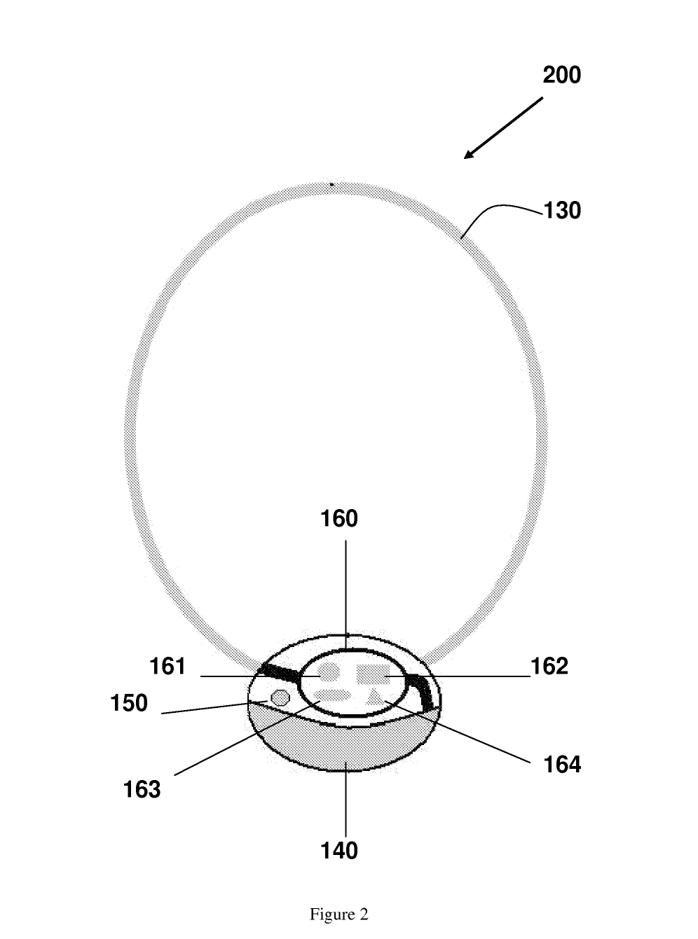

The drawing showed in FIG. 1 is one embodiment of present invention, which is a non-stuck necklace-type detector 100 having a working electrode 140, a temperature sensor 150, a computing center 160 (contains signal processing unit 161, data storage unit 163, and wireless transceiver 164), a battery 162, and a reference electrode 110, wherein the former four components are placed in the pendant part and the last one component is a metal plate placed on the chain. FIG. 2 is another embodiment of this invention which has minor different with FIG. 1 on the design of reference electrode; the reference electrode 130 in FIG. 2 is integrated into the chain. In this example, the working electrode is an induction coil placed inner the pedant.

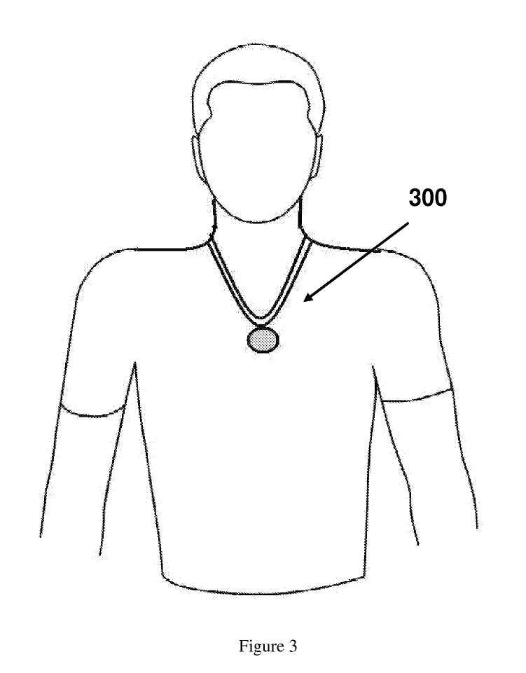

As FIG. 3 shows, this detector 300 is worn on a user's neck and the pendant hangs in front of chest. When doing a measurement, the user is asked to keep steady posture and let the detector fixed onto his skin for a few seconds, then the electrocardiographic signal can be got from detecting the chest surface voltage by the working electrode 140, and the temperature information will also be gained by the temperature sensor 150.

To improve the convenience and reliability of electrocardiography measurement, a two-electrode system is adopted, but this system has more noise problem than a three-electrode system, thus we need some appropriate filter circuit and optocoupler to overcome this defect. For example, in this invention we use an amplification device (a R.O.C. patent of the same inventor, patent No. 149299, application date Jan. 15, 1998) to amplify the electrocardiographic signal detected by the working electrode and to get a useful wave pattern of the signal-to-noise ratio.

The present invention also has another function on body temperature measurement. The detection principle is as same as any commercial electric thermometer that detects a voltage change, resistance change or current change on the inner circuit of the sensor caused by heat transmission, and converts this information into electric signals

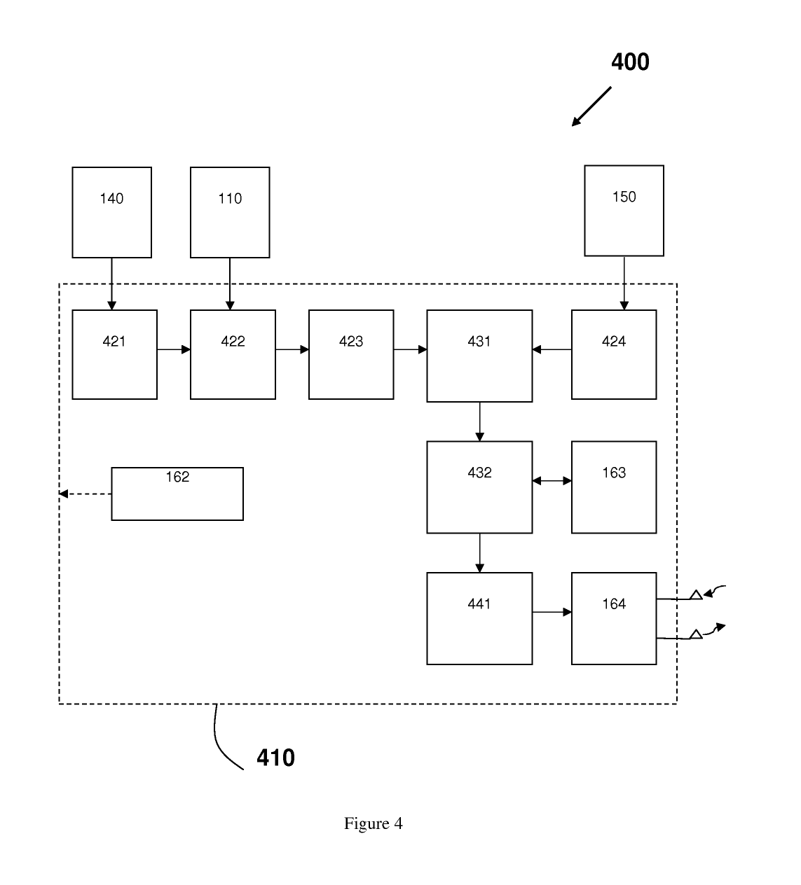

The FIG. 4 shows a diagram of the circuit design 400 of the present invention. The electrocardiographic signals got from the working electrode 140 first pass through an input filter 421 to enhance signal-to-noise ratio, then the signals are amplified by an amplifier 422 which uses the reference electrode 110 as a standard, and then the signals pass through an outpour filter 423 in order to facilitate the sampling. The body temperature signals also pass through a filter/amplify unit 424 to be filtered and amplified. These two signals are then converted into digital signals after passing through an analog-to-digital converter (ADC) 431 and processed in a micro arithmetic logic unit 432, and then either storied in a memory unit 163 or pass through a modulator/demodulator 441 to further transmit to a far-end computer through the wireless transceiver 164. The wireless transceiver 164 also can receive the feedback signal sent from the far-end receiver. A power supply 162 gives power to this signal processing unit 410.

There are many useful wireless interfaces for signal transmission at present such as radio wave and infrared rays. In this example we adopt the Infrared Data Association (IrDA) interface for signals transmission and the far-end computer has an infrared port (IR port) for receive the signals sent by this detector and has the ability to process those analysis listed below:

[Processing of ECG signals] The computer program for HRV analysis was described in our previous publication (Kuo et al. 1999, American Journal of Physiology 277: H2233-H2239.). In the QRS identification procedure, the computer first detected all peaks of the digitized ECG signals using a spike detection algorithm similar to general QRS detection algorithms. Parameters such as amplitude and duration of all spikes were measured so that their means and standard deviations (SD) could be calculated as standard QRS templates. Each QRS complex was then identified, and each ventricular premature complex or noise was rejected according to its likelihood in standard QRS templates. The R point of each valid QRS complex was defined as the time point of each heart beat, and the interval between two R points (R-R interval) was estimated as the interval between current and latter R points. In the R-R interval rejection procedure, a temporary mean and SD of all R-R intervals were first calculated for standard reference. Each R-R interval was then validated: if the standard score of an R-R value exceeded 3, it was considered erroneous or nonstationary and was rejected. The average percentile of R-R rejection according to this procedure was 1.2%. The validated R-R values were subsequently resampled and interpolated at the rate of 7.11 Hz to accomplish the continuity in time domain.

[Frequency-domain analysis] Frequency-domain analysis was performed using the nonparametric method of fast Fourier transform (FFT). The direct current component was deleted, and a Hamming window was used to attenuate the leakage effect. For each time segment (288 s, 2,048 data points) our algorithm estimated the power spectral density on the basis of FFT. The resulting power spectrum was corrected for attenuation resulting from the sampling and the Hamming window. The power (TP) spectrum was subsequently quantified into various frequency-domain measurements as defined in Table 1. In particular, LF was normalized by the percentage of total power except for VLF (total power VLF) to detect sympathetic influence on HRV (LF %). A similar procedure was also applied to HF (HF %). All HRV parameters were expressed in original, square root, and natural logarithmic form to demonstrate and correct possible skewness.

TABLE 1
Definitions for measurements of heart rate variability

| Variable | Units | Definition | Frequency Range |
|---|---|---|---|
| Variance | ms² | Variance of R-R intervals | |
| | | over temporal segment | |
| VLF | ms² | Power in VLF range | 0.003-0.04 Hz |
| LF | ms² | Power in LF range | 0.04-0.15 Hz |
| HF | ms² | Power in HF range | 0.15-0.4 Hz |
| LF/HF | ratio | LF (ms²)/HF (ms²) | |
| LF % | nu | LF power in normalized units: | |
| | | LF/(total power − VLF) × 100 | |
| HF % | nu | HF power in normalized units: | |
| | | HF/(total power − VLF) × 100 | |

nu, Normalized units;
VLF, very-low frequency;
LF, low frequency;
HF, high frequency.

[DATA decipherment] The data were deciphered according to the report published by American Heart Association Inc. and European Society of Cardiology (Anonymous 1996, Circulation 93: 1043-1065, and simultaneously published in European Heart Journal (1996) 17, 354-381.), and also the inventors' previously publications (Kuo et al. 1999, American Journal of Physiology 277: H2233-H2239; Kuo et al. 1997, American Journal of Physiology 273: H1291-H1298; Yang et al. 2000, American Journal of Physiology-Heart and Circulatory Physiology 278:H1269-1273; Yien et al. 1997, Critical Care Medicine 25: 258-266). The HF and TP value are used for measuring the activity of cardiac sympathetic nerve, LF/HF ratio is used for measuring the activity of cardiac parasympathetic nerve, and LF value is a target for estimating the function of sympathetic and parasympathetic nerves.

One skilled in the art readily appreciates that the present invention is well adapted to carry out the objects and obtain the ends and advantages mentioned, as well as those inherent therein. The necklace-type non-stuck detector for electrocardiographic and temperature information measurement is representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A non-stuck detector for electrocardiographic and temperature information measurement comprising: (a) a working electrode, a reference electrode and a temperature sensor connected by a chain; (b) a signal processing unit for processing those signals got from said electrode/sensor, (c) a wireless transceiver for sending/receiving messages to/from a far end; and (d) a power supply.

2. The detector according to claim 1, wherein the working electrode and the reference electrode are constructed into a two-electrode system, the former detects a surface voltage of a use's skin and the latter provides a reference voltage, thus an electrocardiographic signal is got by comparing the surface voltage and the reference voltage.

3. The detector according to claim 2, wherein the working electrode comprises an induction coil to detect the surface voltage of the user's skin.

4. The detector according to claim 1, wherein the reference electrode is applied on detection and amplification of electrocardiographic signals.

5. The detector according to claim 1, wherein the working electrode and the reference electrode are placed separately on the chain part and a pendant part linked to said chain.

6. The detector according to claim 5, wherein the electrode placed on the chain part is the chain itself or is a conductive plate on the chain.

7. The detector according to claim 5, wherein the electrode placed on the pendant is a conductive outer shell of said pendant or is an induction coil underneath the shell.

8. The detector according to claim 7, wherein the conductive outer shell is made by metal or plastic having electrical conductivity.

9. The detector according to claim 8, wherein the plastic is conductive polymer, or is polymer processed by electroplating or coating with conductive material.

10. The detector according to claim 1, wherein the temperature sensor detects body temperature through resistance thermometer, thermistor, thermocouple, or any non-invasive electric-thermometer.

11. The detector according to claim 1, wherein the signal processing unit comprising: (a) a signal amplifier; (b) an electronic filter; and (c) an analog-to-digital converter (ADC).

12. The detector according to claim 11, wherein the electronic filter is selected from the group consisting of low-pass filter, high-pass filter, band-pass filter, band-stop filter and all-pass filter.

13. The detector according to claim 1, wherein the wireless transceiver transports signals through radio wave interface or through infrared interface.

14. The detector according to claim 13, wherein the radio wave interface is IEEE 802 standard, RFID, GSM, PHS or CDMA.

15. The detector according to claim 1, wherein the power supply is battery.

16. The detector according to claim 1, wherein the signal processing unit, the wireless transceiver and the power supply are placed in the pendant.

17. The detector according to claim 1, which further comprises a data storage unit.

18. The detector according to claim 17, wherein the data storage unit is a component for electrical signals storage.

19. The detector according to claim 1, which is wore on user's neck, wrists or limbs.
